(19) United States
(12) Patent Application Publication
(10) Pub. No.: US 2005/0213700 A1
(43) Pub. Date: Sep. 29, 2005

(54) Imaging tomography apparatus with out-of-balance compensation

(76) Inventors: Gunter Danz, Gross-Zimmern (DE); Hans-Jurgen Muller, Pretzfeld (DE)

Correspondence Address: SCHIFF HARDIN LLP;Patent Department, 6600 Sears Tower, 233 South Wacker Drive, Chicago, IL, 60606, US

(21) Appl. No.: 11/045,893

(22) Filed: Jan. 28, 2005

(30) Foreign Application Priority Data
Jan. 28, 2004 (DE) 10 2004 004 301.9

(51) Int. Cl. G21K 1/12; A61B 6/00; H05G 1/60; G01N 23/00; E21B 7/04

(52) U.S. Cl. 378/4

(57) ABSTRACT

An imaging tomography apparatus, in particular an x-ray tomography apparatus or an ultrasound tomography apparatus, has a stationary unit with a measurement unit for measurement of an out-of-balance condition, on which stationary unit is mounted an annular measurement device rotatable around a patient tunnel. Compensation weights for compensation of the out-of-balance condition are provided on the measurement device. To simplify the balancing procedure, the compensation weights are fashioned in the form of compensation rings surrounding the patient opening and with respectively defined out-of-balance conditions. The compensation rings are mounted on the measurement device such that they can be varied with regard to their relative positions in two parallel planes axially separated from one another.

### BACKGROUND OF THE INVENTION

1. Field of the Invention

The invention concerns an imaging tomography apparatus, in particular an x-ray computed tomography apparatus.

2. Description of the Prior Art

An x-ray computed tomography apparatus is known from German OS 101 08 065. A data acquisition device or gantry, mounted such that it can be rotated around a horizontal rotational axis, is accommodated in a stationary mount. A sensor to detect an out-of-balance (unbalanced) condition of the data acquisition device is provided on the stationary mount. The sensor is connected with a device to calculate the position or positions of the rotatable data acquisition device at which a compensation weight or weights should be applied to compensate the out-of-balance condition. The balancing can ensue without the use of a specific balancing device, but a trained person is required to implement the balancing procedure, in particular for correct application of the compensation weights. The balancing procedure requires, among other things, a partial demounting of parts of the x-ray computed tomography apparatus. This procedure thus is time-consuming and expensive.

U.S. Pat. No. 6,354,151 as well as German Translation 698 04 817 T2 describe an apparatus for balancing of an instrument mounting. The mass of the instrument mounting and its out-of-balance condition are thereby determined.

German Utility Model 297 09 273 discloses a balancing device for balancing rotors. Two compensation rings with a defined out-of-balance condition are provided that can be attached to one another on the rotor at suitable relative positions for compensation of an out-of-balance condition of the rotor.

German PS 199 20 699 also discloses a method for balancing rotors. Two compensation rings respectively exhibiting a defined out-of-balance condition are mounted on the rotor. To compensate the out-of-balance condition, the relative positions of the compensation rings relative to one another can be changed. For this purpose, an attachment device of the compensation rings is released. The compensation rings are held by a pawl and the rotor is rotated by a predetermined angle relative to the compensation rings. The compensation rings are subsequently locked (arrested).

To ease the locking of such compensation rings, in German OS 199 20 698 it is disclosed to fix the rings in their relative positions by means of a spring-loaded locking device on the rotor. By means of an applied force, the compensation rings can be displaced in their relative positions relative to the rotor and naturally can be locked.

To ease the identification of the correct locking position of such compensation rings, in German Utility Model 298 23 562 discloses projecting markings onto the compensation elements by means of a marking device when the rotor is located in a compensation position.

German PS 197 29 172 discloses a method for continuous compensation of an out-of-balance rotor. The out-of-balance condition of the rotor is measured by means of an out-of-balance measurement device. For compensation of the out-of-balance condition, the rotor has a number of compensation chambers filled with compensation fluid and disposed at different relative rotor positions. To compensate the out-of-balance condition, the quantity of the compensation fluid in the compensation chambers is increased or reduced in a suitable manner.

German Utility Model 299 13 630 concerns an apparatus for compensation of an out-of-balance condition in a machine tool or balancing machine. The balancing machine is thereby balanced using counterweight rotors and the position of the counterweight rotors is stored. The balancing machine is subsequently re-balanced with a component incorporated therein by displacement of the counterweight rotors. The out-of-balance condition of the component can be inferred from the deviating position of the counterweight rotors without and with the component.

German OS 197 43 577 and German OS 197 43 578 disclose a method for balancing a rotating body. Compensation masses that can be radially displaced and/or displaced in terms of their relative positions with respect to the rotating body are attached to the rotating body. At the beginning of the method, the compensation masses are initially brought into a zero position in which the vectors generated by them mutually cancel. The out-of-balance condition of the rotating body is subsequently measured and compensated by suitable shifting of the compensation masses.

The implementation of these known methods typically requires technically trained personnel. Independently of this, some of the known methods are not suited for balancing of a measurement device of a tomography apparatus.

### SUMMARY OF THE INVENTION

An object of the present invention to remedy the aforementioned disadvantages according to the prior art. In particular, an imaging tomography apparatus should be provided having a rotatable measurement device that can be optimally simply balanced. The balancing procedure should be fully automatically implementable, such that trained personnel are not required.

This object is achieved according to the invention by an imaging tomography apparatus having a data acquisition device mounted for rotation around a patient opening of a stationary unit, wherein compensation weights are fashioned in the form of compensation rings with respective defined out-of-balance conditions, the compensation rings surrounding the patient opening, and the compensation rings are mounted on the data acquisition device in two parallel planes that are separated from one another such that the compensation rings can be varied with regard to their relative positions.

An out-of-balance condition of the data acquisition device can thus be compensated in a particularly simple manner, namely by a rotation of the compensation rings relative to the data acquisition device. The compensation can ensue completely automatically. Because the compensation weights are arranged in two parallel planes axially separated from one another, a comprehensive compensation of axial and radial out-of-balance vectors is possible.

A further measurement unit is provided to determine the rotation angle of the data acquisition device. This enables an exact determination of the relative positions or the position of the compensation weights on the data acquisition device as well as an automatic shifting thereof into a new position.

Each of the compensation rings can be adjustable in terms of its relative position with regard to the data acquisition device by means of a motor. By a suitable activation of the motors, a completely automatic balancing of the data acquisition device is possible. The balancing can even ensue during the operation of the data acquisition device. In addition, it is possible to adjust the compensation rings electromagnetically. For this purpose, reference is made to German OS 43 37 001, the teachings of which are incorporated herein by reference.

To control the motors according to a predetermined algorithm for compensation of an out-of-balance condition, a control device is provided. Such a control device is, for example, a conventional controller with a microprocessor. The control device can be connected with a sensor that measures the out-of-balance condition as well as with a further sensor that determines the rotational angle of the data acquisition device. Control signals for rotation of the compensation rings by a predetermined angle amount relative to the data acquisition device can be generated with the control device. A completely automatic balancing of the data acquisition device is thus possible. Trained personnel are not necessary for this.

In an embodiment, two compensation rings are associated with each of the aforementioned parallel planes. This enables a balancing in each plane according to a technique known as the expansion angle method. For this, the relative position of the compensation rings relative to one another is adjusted in a suitable manner in each of the two planes.

At least one of the compensation rings can be attached between a detector provided on the data acquisition device and a slip ring. In this case, the slip ring is axially separated from the detector. This enables a compact structural shape.

An inner radius of the compensation rings can approximately correspond to an inner radius of the data acquisition device. In this case, an outer radius of the compensation rings is typically smaller than an outer radius of the data acquisition device. In this case, the compensation rings are attached in proximity to the inner radius. Alternatively, an outer radius of the compensation rings may approximately correspond to an outer radius of the data acquisition device. In this case, an inner radius of the compensation rings can be larger than an inner radius of the data acquisition device. In this case, the compensation rings are attached in the region of the outer radius of the data acquisition device.

It has proven to be advantageous to attach the compensation rings to the data acquisition device by means of thin ring bearings, such that they can rotate. This saves space and enables a compact design of the data acquisition device.

### DESCRIPTION OF THE DRAWINGS

FIG. 1 is a schematic side view of an x-ray tomography apparatus.

FIG. 2 is a schematic representation of the compensation rings in accordance with the invention.

FIG. 3 is a schematic axial section through a first measurement device in accordance with the invention.

FIG. 4 is a schematic axial section through a second measurement device in accordance with the invention.

### DESCRIPTION OF THE PREFERRED EMBODIMENTS

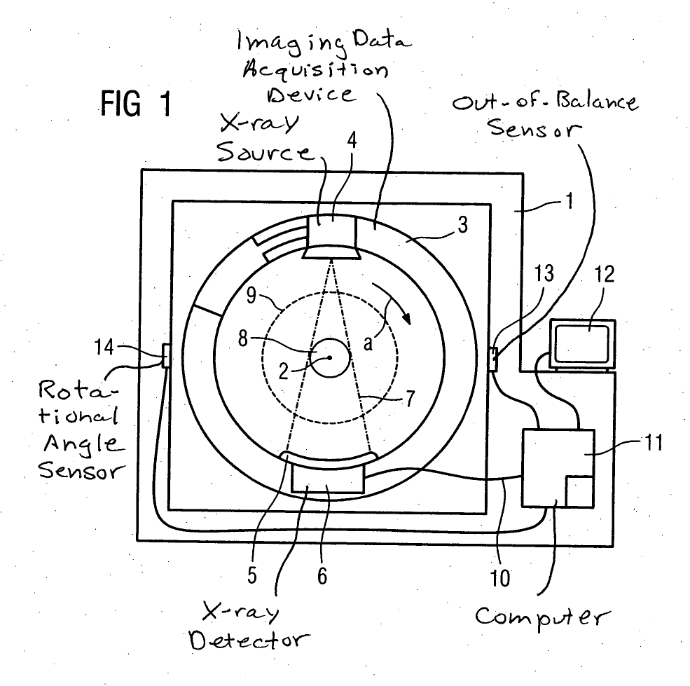

FIG. 1 schematically shows a side view of an x-ray tomography apparatus with a stationary unit 1. An annular imaging data acquisition device 3 (gantry) is accommodated on the stationary unit 1 such that it can rotate around a rotation axis 2 disposed at a right angle to the plane of the drawing. The rotation direction of the imaging data acquisition device 3 is designated with the arrow a. An x-ray source 4 and an x-ray detector 5 with downstream evaluation electronic 6 are mounted on the imaging data acquisition device 3 opposite to each other. A beam fan 7 radiated by the x-ray source 4 defines a circular measurement field 8 given a rotation of the imaging data acquisition device 3. The measurement field 8 is located within a patient opening 9 indicated with the dashed line. The evaluation electronic 6 is connected with a computer 11 via a slip ring contact 10 (indicated schematically). The computer 11 has a monitor 12 for display of data. A sensor 13 for measurement of vibrations transferred to the stationary unit 1 is provided on the stationary unit 1. This is a conventional sensor with which vibrations caused by an out-of-balance condition of the imaging data acquisition device 3 and transferred to the stationary unit 1 can be measured in the radial direction and the axial direction. A further sensor 14 attached to the stationary unit 1 serves for the detection of the rotational angle of the imaging data acquisition device 3 relative to the stationary unit 1. The sensor 13 and the further sensor 14 are likewise connected with the computer 11 for evaluation of the signals measured therewith. In FIG. 1, for clarity compensation rings provided on the data acquisition device 3 are not shown.

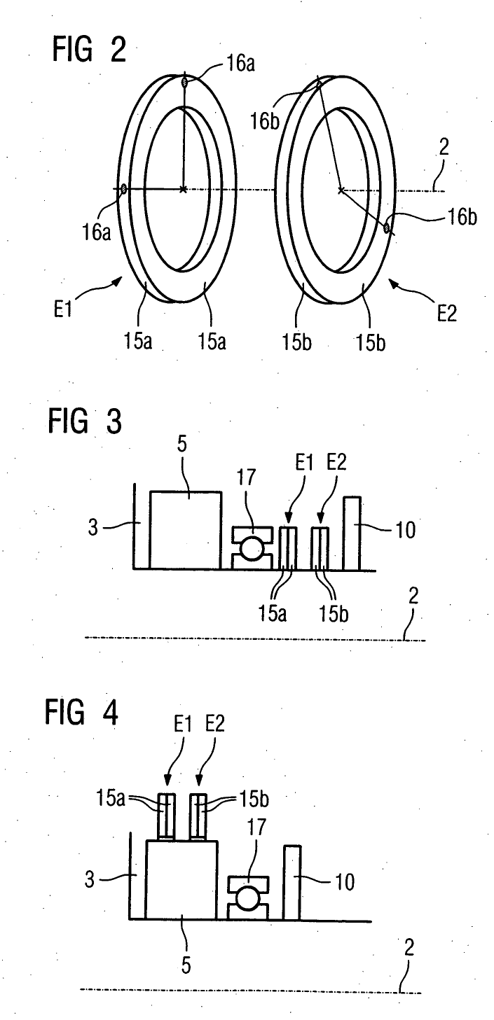

In the schematic representation shown in FIG. 2, two first compensation rings 15a immediately adjacent each other in a first plane E1 and two second compensation rings 15b likewise immediately adjacent each other in a second plane E2 are disposed so that they can rotate around the rotation axis 2. Each of the compensation rings 15a, 15b exhibits a predetermined out-of-balance condition. Additionally, the first compensation rings 15a are provided with first compensation weights 16a and the second compensation rings 15b are provided with second compensation weights 16b. Each of the first compensation rings 15a and the second compensation rings 15b can be connected with a motor (not shown) such it can be driven thereby. The compensation rings 15a, 15b are attached to the data acquisition device 3 (not shown) and are adjustable around the rotation axis 2 in terms of their relative position relative to the data acquisition device by means of the motors.

FIG. 3 schematically shows a partial cross-sectional view of a first embodiment of the data acquisition device 3. The data acquisition device 3 is accommodated on the stationary unit (not shown) such that it can rotate around the rotational axis 2 by means of a bearing 17. The slip ring 10 is arranged on one end of the data acquisition device 3 for power supply as well as for transfer of data. Located between the x-ray detector 5 and the collector ring 10 in a first plane E1 and a second plane E2 are the first compensation rings 15a and the second compensation rings 15b arranged in pairs. The first plane E1 and the second plane E2 are separated parallel and axial to one another. An inner radius of the compensation rings 15a, 15b approximately corresponds to the inner radius of the data acquisition device 3.

In the second embodiment of the data acquisition device 3 shown in FIG. 4, the compensation rings 15a, 15b surround the x-ray detector 5 and an oppositely disposed x-ray source (not shown). An outer radius of the compensation rings 15a, 15b here approximately corresponds to the outer radius of the data acquisition device 3.

Naturally, other arrangements of the compensation rings 15a, 15b are possible. The compensation rings 15a, 15b can be arranged, for example, to the left and right next to the x-ray detector 5. Alternatively, for example, the first compensation rings 15a can surround the x-ray detector 5 and the x-ray source, in contrast to which the second compensation rings 15b are arranged to the left or right next to the bearing 17.

Two sensors 13 (only one of which is shown in FIG. 1) are mounted on the stationary unit 1 to measure vibrations exerted on the stationary unit 1 by an out-of-balance condition of the data acquisition device 3, with one sensor 13 for each plane E1, E2. The sensors 13 are appropriately arranged on the stationary unit 1 with a displacement (offset) of 90° with regard to the rotational axis 2. This enables the determination of radial out-of-balance vectors of each plane E1, E2 in a particularly simple manner, and thus allows a particularly comprehensive compensation of the out-of-balance condition of the data acquisition device 3.

The functioning of the tomography apparatus is as follows:

Initially, the compensation rings 15a, 15b in each plane E1, E2 are located in a null position in which the out-of-balance vectors cancel each other. The first compensation weights 16a of the first compensation rings 15a are displaced by an angle of approximately 90° with regard to the rotational axis 2. The second compensation weights 16b of the second compensation rings 15b are displaced with regard to the first compensation weights 16a by an angle of approximately 180° with regard to the rotation axis 2. An arrangement of the compensation weights 16a, 16b with a displacement of respectively approximately 90° results in an axial projection.

The data acquisition device 3 is rotated. The vibrations transferred to the stationary unit 1 due to the out-of-balance condition of the first data acquisition device 3 are measured by the first sensors 13. The rotational angles of the data acquisition device 3 relative to the stationary unit 1 are simultaneously registered by the second sensor 14. Using a suitable calculation program (algorithm) stored in the computer 11, positions or corresponding angles for the compensation weights 16a, 16b suitable for compensation of the out-of-balance condition of the data acquisition device 3 are respectively calculated for both planes E1, E2. The compensation rings 15a, 15b are subsequently adjusted in each of the two planes E1, E2 by the thus determined angles relative to the data acquisition device 3, such that the out-of-balance condition of the data acquisition device 3 is compensated.

The method can be implemented automatically. Trained personnel are not necessary for this.

Although modifications and changes may be suggested by those skilled in the art, it is the intention of the inventors to embody within the patent warranted hereon all changes and modifications as reasonably and properly come within the scope of their contribution to the art.

1. An imaging tomography apparatus comprising:
a stationary unit having a patient opening therein;
an annular imaging data acquisition device rotatably mounted in said stationary unit for rotation around said patient opening;
a first sensor disposed with respect to said imaging data acquisition device for measuring an out-of-balance condition of said imaging data acquisition device;
a second sensor disposed with respect to said imaging data acquisition device for measuring a rotational angle of said imaging data acquisition device;
a plurality of compensation rings, forming compensation weights, surrounding said patient opening, each of said compensation rings having its own defined out-of-balance condition, said compensation rings being mounted on said imaging data acquisition device allowing adjustment of said compensation rings with regard to their relative positions in two parallel planes in said imaging data acquisition device axially separated from each other;
a motor in driving connection with said plurality of compensation rings for adjusting said relative positions of said compensation rings; and
a control device connected to said first sensor, said second sensor and said motor, said control device controlling said motor to adjust said relative positions of said compensation rings according to a compensation algorithm, employing said measurement of said out-of-balance condition of said imaging data acquisition device and said rotational angle of said imaging data acquisition device, to compensate out-of-balance condition of said imaging data acquisition device.

2. An imaging tomography apparatus as claimed in claim 1 wherein said imaging data acquisition device is an x-ray tomography data acquisition device.

3. An imaging tomography apparatus as claimed in claim 1 wherein said imaging data acquisition device is an ultrasound tomography device.

4. An imaging tomography apparatus as claimed in claim 1 wherein said plurality of compensation rings consists of four of said compensation rings, with two of said compensation rings being disposed in each of said two parallel planes.

5. An imaging tomography apparatus as claimed in claim 1 wherein said imaging data acquisition device includes an x-ray source and an x-ray detector, said x-ray detector generating detector data dependent on x-rays from said x-ray source that are incident on said x-ray detector, and a slip ring connected to said x-ray detector for conveying said detector data from said imaging data acquisition device to said stationary unit, and wherein at least one of said compensation rings is disposed between said x-ray detector and said slip ring.

6. An imaging tomography apparatus as claimed in claim 1 wherein said imaging data acquisition device has an inner radius, defined by said patient opening, and wherein each of said compensation rings has an inner radius approximating said inner radius of said imaging data acquisition device.

7. An imaging tomography apparatus as claimed in claim 1 wherein said imaging data acquisition device has an outer radius, and wherein each of said compensation rings has an outer radius approximating said outer radius of said imaging data acquisition device.

8. An imaging tomography apparatus as claimed in claim 1 wherein said first sensor is attached to said stationary unit.

9. An imaging tomography apparatus as claimed in claim 1 comprising a plurality of thin ring bearings in which said plurality of compensation rings are respectively mounted, allowing rotation of the respective compensation rings in the respective ring bearings.
